(19) United States
(12) Patent Application Publication
(10) Pub. No.: US 2015/0322114 A1
(43) Pub. Date: Nov. 12, 2015

(54) Self-Assembling Peptides

(71) Applicant: QUEEN MARY UNIVERSITY OF LONDON, London (GB)

(72) Inventors: Alvaro Mata Chavarria, London (GB); Karla Inostroza, London (GB); Katherine Smith, Barcelona (ES); Estelle Collin, London (GB)

(21) Appl. No.: 14/655,090

(22) PCT Filed: Dec. 24, 2013
(86) PCT No.: PCT/GB13/53432

§ 371 Date: Jun. 24, 2015

Related U.S. Application Data
(60) Provisional application No. 61/745,669, filed on Dec. 24, 2012.

(51) Int. Cl. C07K 14/00

(52) U.S. Cl. CPC C07K 14/001

(57) ABSTRACT

There is provided a composition comprising a self-assembling membrane, wherein the self-assembling membrane comprises elastin-like polymers (ELPs) and peptide amphiphiles (PAs). Also provided is a method of forming a membrane and uses of the membrane.

### FIELD OF THE INVENTION

The present invention relates to peptides which are capable of self-assembling into membranes that can spontaneously undergo morphogenesis. In particular, peptide amphiphiles (PAs) and elastin-like polymers (ELPs), when mixed together, assemble into a hybrid anisotropic membrane that can open. These membranes can be easily formed into tubes, tubular networks and other structures.

### BACKGROUND TO THE INVENTION

Molecular self-assembly is one of the most promising strategies for developing the next generation of functional materials. This process allows nature to create a widespread variety of well-ordered materials made from multiple building-blocks. Human tissues, for example, are composed of an assortment of well-organized macromolecules and proteins that give rise to hierarchical structures with amazing properties and function. However, such complex structures require more than just well-organized building-blocks, but rather dynamic and well-controlled mechanisms that direct self-assembly with precise spatiotemporal control.

Peptides and proteins have received much attention in the field of biomaterials owing to the relative ease with which they can be produced, their inherent functionality, and the increased capacity to manipulate them through self-assembling mechanisms. The potential of this material synthesis approach has motivated the generation of highly sophisticated self-assembling systems that are beginning to mimic the complexity and functionality of natural macromolecules. For example, ionic self-complementary peptides with alternating hydrophilic and hydrophobic residues, which upon a change in pH or the addition of salts, self-assemble into 3D nanofibrous gels or small Fmoc peptides that are selectively self-assembled into nanofibres by enzymatic assembly. Peptide-based amphiphilic molecules, either comprising solely amino acids as the hydrophilic and hydrophobic sections, or including an artificial moiety such as an alkyl chain, have been designed to assemble into many different biomimetic nanostructures including vesicles, fibres, belts, and tubes in response to an appropriate cue. Likewise, molecules based on proteins like silk, collagen or elastin can also be used to form biomimetic functional structures. Recombinant elastin-like peptides (ELPs) are a type of protein-like molecules that consist of repeating pentapeptide sequences of VPGXG (SEQ ID NO: 1), where X is any amino acid apart from proline. These molecules undergo a phase transition at a certain transition temperature (Tt) and reversibly assemble into hydrogels. Their Tt can be easily altered by varying the amino acid sequence and bioactivity can also be added via the incorporation of specific peptide domains.

Whilst these sophisticated systems give rise to extremely valuable materials for many different applications, gaining hierarchical control over their macroscopic structure would provide greater functionality. To this end, attempts have been reported using soft lithographic techniques to provide anisotropic structure to ELP and PA materials. Combining a thermal self-assembling pathway with an extrusion step has recently been reported to produce macroscopic noodle-like or tubular gels made from long range aligned nanofibres. In addition to controlled hierarchy, the addition of multiple building-blocks would significantly improve complexity and versatility. A particularly interesting approach has been developed by co-assembling PAs with oppositely charged biopolymers such as hyaluronic acid or alginate. This system has been used to generate macroscopic sacs, cell-like microcapsules, and robust bioactive membranes. Motivated by this approach, similar hybrid membranes have been produced solely by charge interactions using commercially available surfactant molecules and oppositely charge polyelectrolytes and topographically micropatterned ones by combining HA with RGD-containing multidomain peptides.

### SUMMARY OF THE INVENTION

The capacity to further guide self-assembly at multiple scales with significant spatiotemporal control to fabricate complex peptide-based structures is still limited. Therefore, aiming to improve on the complexity and hierarchical control of peptide-based materials, the inventors have developed a multiple peptide-based system that allows directed self-assembly on demand to fabricate hybrid hierarchical structures.

In a first aspect, the present invention provides a composition comprising a self-assembling membrane, wherein the self-assembling membrane comprises elastin-like polymers (ELPs) and peptide amphiphiles (PAs).

The ELPs and PAs are oppositely charge so that they interact to form a membrane. This occurs without any external interaction so that the membrane self-assembles. This allows peptide material morphogenesis to form complex geometries by simple self-assembly without the use of templates or moulds.

Elastin-like polymers (ELPs) are proteinaceous polymers which are based on the recurrence of certain short monomers that are considered as “building blocks” in natural elastin due to their striking repetition with no or little variation along its sequence. ELPs are normally rich in amino acids such as glycine (G), valine (V) and proline (P). ELPs are generally based on an amino acid pentamer as the monomer. For example, many ELPs are based on the pentamer VPGVG (SEQ ID NO: 2) (as monomer) or modifications of this in which one or more amino acids have been substituted by other natural or modified ones. Therefore, preferably, the ELPs comprise a polymer of the pentamer VPGVG (SEQ ID NO: 2) and/or a pentamer in which one or more (e.g. two or three) amino acids have been substituted by other natural or modified ones. The ELPs may comprise a polymer formed from more than one pentamer.

The ELPs are charged. They may be negatively charged or positively charged. In some embodiments, the ELPs are negatively charged. In other embodiments, the ELPs are positively charged. The charge polarity should be opposite to that of the PAs. The polymer may comprise a pentamer which contains a negatively charged amino acid such as aspartic acid (D) or glutamic acid (E). The polymer may comprise a pentamer which contains a positively charged amino acid such as histadine, lysine or arginine.

The ELPs may comprise a plurality of hydrophobic regions. This helps in the self-assembly of the membrane. It also allows an opening to be formed in the membrane which allows the formation of tube geometries. For example, the tube with three or more openings can be formed.

The ELPs have a critical temperature (transition temperature—Tt) at which phase transitional behaviour occurs. Below this temperature, the polymer chains remain disordered, relatively extended, in solution if they are not crosslinked, and fully hydrated mainly by hydrophobic hydration. On the contrary, above Tt, the polymer chain hydrophobically folds and assembles to form a phase separated state in which the chains adopt a dynamic, regular, non-random structure, called p-spiral, involving one type II β-turn per pentamer, and stabilized by intraspiral inter-turn and inter-spiral hydrophobic contacts. In some embodiments, the ELPs have a Tt of between about 0° C. and about 20° C. In other embodiments, the ELPs have a Tt of between about 0° C. and about 15° C.

Particular pentamers which can be used in the formation of an ELP include but are not limited to VPGVG (SEQ ID NO: 2). VPGDG (SEQ ID NO: 3). VPGEG (SEQ ID NO: 4), VGIPG (SEQ ID NO: 5) and VPGIG (SEQ ID NO: 6). In some embodiments, the ELPs comprise a polymer comprising, as a monomer, one or more pentamer selected from VPGVG (SEQ ID NO: 2), VPGEG (SEQ ID NO: 4), VGIPG (SEQ ID NO: 5) and VPGIG (SEQ ID NO: 6). In other embodiments, the ELPs comprise a polymer comprising, as monomers, two or more pentamers selected from VPGVG (SEQ ID NO: 2). VPGEG (SEQ ID NO: 4), VGIPG (SEQ ID NO: 5) and VPGIG (SEQ ID NO: 6). In further embodiments, the ELPs comprise a polymer comprising, as monomers, three or more pentamers selected from VPGVG (SEQ ID NO: 2), VPGEG (SEQ ID NO: 4). VGIPG (SEQ ID NO: 5) and VPGIG (SEQ ID NO: 6). In particular embodiments, the ELPs comprise a polymer comprising, as monomers, the pentamers VPGVG (SEQ ID NO: 2), VPGEG (SEQ ID NO: 4), VGIPG (SEQ ID NO: 5) and VPGIG (SEQ ID NO: 6).

The polymer may contain the pentamers in the following ratios relative to each other:
a) VPGVG: 60-100;
b) VPGEG: 10-30;
c) VGIPG: 100-140; and
d) VPGIG: 20-60.

It has been found that ELPs with these ratios of components are especially good at forming self-assembling membrane.

In some embodiments, the polymer contains the pentamers in the following ratios relative to each other:
a) VPGVG: 70-90;
b) VPGEG: 10-30;
c) VGIPG: 110-130; and
d) VPGIG: 30-50.

In other embodiments, the polymer may contain the pentamers in the following ratios relative to each other:
a) VPGVG: 75-85;
b) VPGEG: 15-25;
c) VGIPG: 115-125; and
d) VPGIG: 35-45.

The ELPs may comprise additional components as part of the polymer in addition to the monomers (e.g. pentamers). For example, the ELPs may comprise a bioactive domain to elicit a specific biological effect. The ELPs may comprise a plurality of bioactive domains. The bioactive domain may be a peptide sequence. The bioactive domain may cause cell adhesion. For example, the peptide sequence RGD can be inserted into the ELP as a cell adhesion domain.

In some embodiments, the ELP may have the sequence of SEQ ID NO: 7, 8, 9 or 10.

In particular embodiments, a positively charged ELP has the following sequence:

(SEQ ID NO: 7)
[[(VPGIG)₂(VPGKG)(VPGIG)₂]₂AVTGRGDSPASS
[(VPGIG)₂(VPGKG)(VPGIG)₂]₂]₆

In some embodiments, the molecular weight of the ELPs range between about 2 kDa and about 400 kDa. In some embodiments, the molecular weight of the ELPs range between about 5 kDa and about 300 kDa. In other embodiments, the molecular weight of the ELPs range between about 10 kDa and about 250 kDa.

Preferably, the ELPs have an average molecule weight of between about 2 kDa and about 500 kDa. The ELPs may have an average molecule weight of between about 10 kDa and about 50 kDa. In particular embodiments, the ELPs have an average molecule weight of between about 20 kDa and about 40 kDa. In some embodiments, the ELPs have an average molecule weight of about 30 kDa.

Peptide amphiphiles (PAs) are molecules which have a hydrophilic portion provided by a peptide and a hydrophobic portion. Generally, the hydrophobic portion is provided by a hydrophobic tail attached to the peptide. Alternatively, the hydrophobic portion may be a peptide sequence formed from hydrophobic amino acids.

In some embodiments, the hydrophobic tail may comprise one or more alkyl tails (e.g. branched or unbranched: saturated or unsaturated). In some embodiments, the PA is a peptide sequence with an alkyl tail having 4-20 carbons attached thereto. In some embodiments, the alkyl tail may have 10-20 carbons. In particular embodiments, the alkyl tail may have 14-18 carbon atoms.

The peptide sequence of the PA is preferably between 3 and 30 amino acids in length. The peptide sequence of the PA may be between 5 and 20 amino acids in length. In some embodiments, the peptide sequence may be between 5 and 15 amino acids. In particular embodiments, the peptide sequence is between 7 and 11 amino acids, and may be 9 amino acids in length.

The peptide sequence of the PAs is charged. It may be negatively charged or positively charged. In some embodiments, the peptide sequence is negatively charged. In other embodiments, the peptide sequence is positively charged. The charge polarity should be opposite to that of the ELPs. A negative charge may be provided by the presence of one or more negatively charged amino acids such as aspartic acid (D) or glutamic acid (E). A positive charge may be provided by one or more positively charged amino acids such as histadine, lysine or arginine.

The peptide sequence may be able to form a β-sheet. This occurs through interaction between the amino acid residues. This helps in the formation of the self-assembling membrane.

The PAs may be able to self-assemble into fibres (e.g. nanofibres) when in solution. i.e. in the absence of ELPs. This ability is down to the properties of the PAs.

As indicated above, the PAs are able to interact with the ELPs to form a membrane made up of the two components.

In some embodiments, the ELPs and PAs are crosslinked. This can be done, for example, using glutaraldehyde. This helps to increase the strength of the membrane.

The self-assembling membrane can be formed into a variety of shapes. For example, it can be formed into a tube. Tubes can be formed having a number of configurations such as a tube with two or more (e.g. three or four) openings, an elongated tube, and connected tubes attached along one side. Tubular networks can be formed with up to 10 tubes. In some embodiments, tubular networks can be formed with more than 10 tubes. Other configurations can also be formed by manipulating and controlling the membrane as it is self-assembling.

The membrane that opens and can form tubes exhibits an anisotropic composition and includes a higher concentration of ELPs on the outside and higher concentration of PAs on the inside. The opening of the ELP chains by the PAs generates swelling of the membrane or pre-stress, which will allow it to open upon contact with an interface, thanks in part to the stickiness of the higher concentration of ELPs on the outside.

The self-assembling process of the membrane permits the incorporation of further components such as cells, growth factors, enzymes, proteins etc. as long as their concentration is not too high so that it does not disturb the self-assembly between the ELPs and the PAs. This allows the membrane to be functionalised. Therefore, the membrane may further comprise cells, growth factors, enzymes or proteins. In particular embodiments, the membrane further comprises silk fibroin protein or a glycan such as heparan sulphate and chondroitin sulphate.

The ELPs and PAs may be present in the membrane at any suitable ratio such that membrane formation takes place. Preferably, the ratio of ELP molecules to PA molecules is between about 1:5 and about 1:20.

The self-assembly of the membrane provides a novel material platform to create complex peptide-based 3D macroscopic structures by controlled self-assembly for any application that would benefit from having peptide-based materials. Particular applications include:
a. Tissue Engineering: development of scaffolds and biomaterials to create tissues and organs in the lab that may be then implanted into patients or to create biomimetic environments that recreate in vivo environments for in vitro cell and biological studies. Examples may include vascular grafts, peripheral nerve conduits, annulus fibrosus grafts, or any tubular or membranous tissue/organ. Cells, growth factors, enzymes and small molecules can be embedded within the membrane as it self-assembles. In vitro applications include the ability to create more biomimetic tissues for drug discovery, better understand illnesses, or as models to understand phenomenon like tissue morphogenesis during development.
b. Regenerative medicine: development of biomaterials that may be implanted into patients and allow self-assembly to take place in the body to further assemble to or within the host tissue.
c. Device fabrication: The process provides a novel process to fabricate devices based on self-assembly due to the potential to fabricate in 3D and control its formation at multiple scales. Devices may include biosensors, electronic devices,

Therefore, the invention also provides the use of the membrane for the above applications, e.g. tissue engineering, regenerative medicine and device fabrication.

In another aspect of the invention, there is provided a method of forming a membrane, the method comprising contacting ELPs and PAs, wherein interaction between the ELPs and PAs causes formation of the membrane.

The properties of the ELPs and PAs are as described above.

In some embodiments, the ELPs and PAs are in aqueous solution such that separate solutions containing each of the components are brought together. The membrane forms at the interface between the ELP and PA solution. The two components should remain localised so that the membrane can form properly in the region between the two localised components. The components should not be mixed.

In particular embodiments, a solution of PAs is added to a solution of ELPs. A solution of PAs may be added to a larger volume of an ELP solution. In this way, the PA solution is initially contained in the ELP solution. For example, the PA solution may be added as a drop into the interior of the ELP solution. This causes the membrane to form between the PA solution and the ELP solution so that the membrane separates and forms a barrier between the two solutions.

In some embodiments, additional PA solution may be added to the ELP solution. This may be in the same region as the first addition so that the PA solution is added to the PA solution which is already present. Alternatively, the additional PA solution may be added to the ELP solution in a different place. This can be used to manipulate the formation of the membrane. For example, if the additional PA solution is added once the membrane has already partially formed, it is possible to produce two membrane structures which are joined together. Two tubes joined along one side can be formed in this way.

The concentration of the PA solution can be any suitable concentration as long as the PAs are completely dissolved and as long as membrane can form when it interacts with ELPs. In some embodiments, the PA solution has a concentration of between about 0.05 wt % and about 5 wt %. In further embodiments, the PA solution has a concentration of between about 0.5 wt % and about 3 wt %.

The concentration of the ELP solution can be any suitable concentration as long as the ELPs are completely dissolved and as long as membrane can form when it interacts with PAs. In some embodiments, the ELP solution has a concentration of between about 0.1 wt % and about 10 wt %. In further embodiments, the ELP solution has a concentration of between about 0.2 wt % and about 2 wt %. In other embodiments, the ELP solution has a concentration of between about 0.5 wt % and about 1.5 wt %.

The membrane can be formed at any suitable pH. Preferably, the membrane is formed at a pH of between about 3 and about 10. In some embodiments, the membrane is formed at a pH of between about 4 and about 9. In other embodiments, the membrane is formed at a pH of between about 5 and about 8. In particular embodiments, the membrane is formed at a pH of between about 6 and about 7.

The membrane may be formed at any suitable temperature. Preferably, the membrane is formed at a temperature which is the same as or greater than the transition temperature (Tt) of the ELPs. In some embodiments, the membrane is formed at a temperature which is greater than the transition temperature (Tt) of the ELPs. For example, if the Tt of the ELPs is 15° C., the membrane may be formed at a temperature of greater than 15° C.

The method should be carried out for a sufficient length of time in order to allow the membrane to form. The PAs and ELPs should be allowed to interact for at least 10 seconds. In order to produce a stronger membrane, the PAs and ELPs should be allowed to interact for a longer period of time. In some embodiments, the PAs and ELPs can be allowed to interact for 12 hours or more. In other embodiments, the PAs and ELPs can be allowed to interact for 24 hours or more. In particular embodiments, the PAs and ELPs can be allowed to interact for up to 48 hours.

When the membrane is forming, it can be manipulated so as to control the shape into which the membrane forms. For example, it can be manipulated so that it forms into tubes having three or more openings. This can be done by touching the wall of a forming tube so that an additional opening is created.

Once the membrane has been formed, the method may further comprise the step of crosslinking the membrane. This can be done by applying a crosslinking agent (e.g. glutaraldehyde) to the membrane.

Once the membrane has formed, it can be embedded in a hydrogel.

The invention also provides a kit for forming a self-assembled membrane, the kit comprising ELPs and PAs.

In the above description, when the term ‘about’ is used in connection with a numerical value, this means that the numerical value can vary by plus or minus 5%. Therefore, the expression ‘about 100’ is equivalent to 100±5 which is equivalent to 95-105.

### DETAILED DESCRIPTION OF THE INVENTION

The invention will now be described in detail, by way of example only, with reference to the following figures:

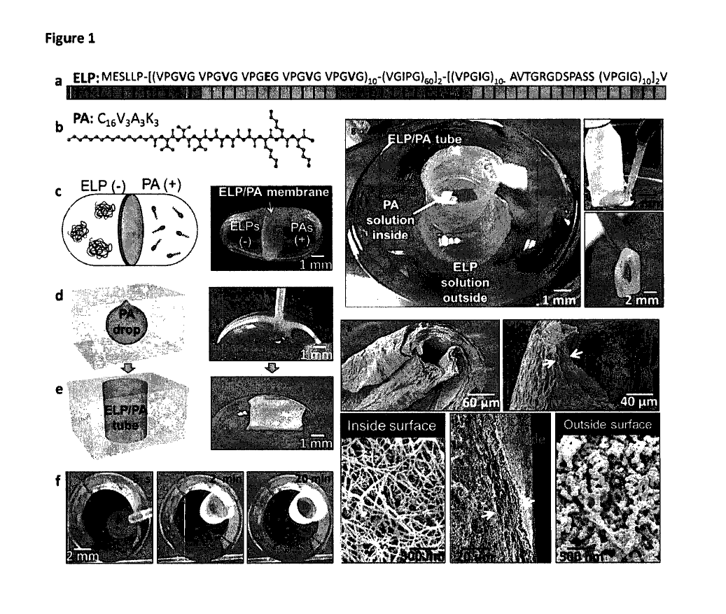

FIG. 1. Co-assembly of elastin-like polymers (ELPs) with peptide amphiphiles (PAs) into tubular macrostructures. a, b, Amino acid sequences for ELPs and PAs used in this study. c-e, Schematic and corresponding images of c, a solid self-assembled membrane upon contact between a solution of PAs and ELPs, d, the initial drop of PAs embedded within a solution of ELPs, e, and the spontaneous formation of a tube structure. f, Time-lapse images of a drop of PAs embedded within the ELP solution and subsequent opening within a few minutes. g, Images of robust and elastic tube structures formed after 48 hr. h, SEM images showing the cross-section of the tubular macrostructure made from a network of nanofibres that exhibit different morphology on the outside and the inside walls of the walls of the tube.

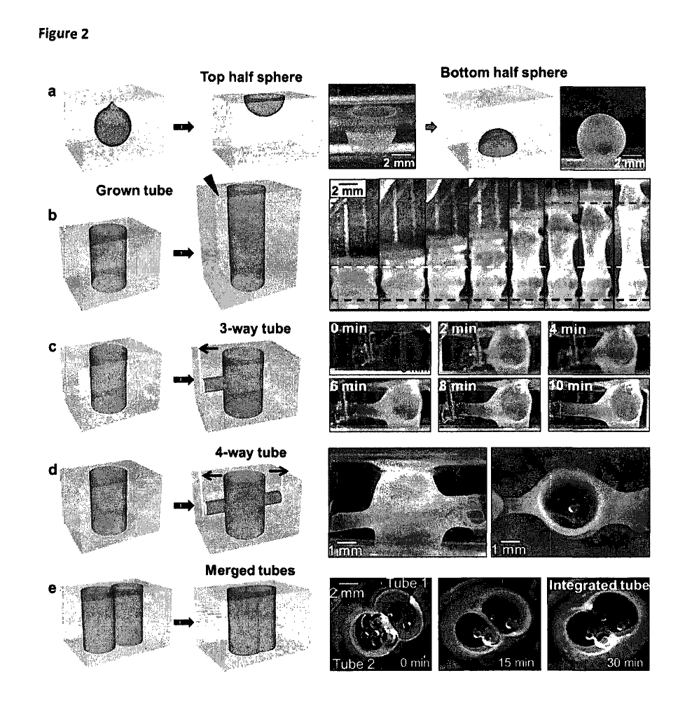

FIG. 2. Manipulation and morphogenesis of the ELP-PA co-assembling system. a, Closed membranes exhibiting only one opening upon contact with either the bottom substrate or the liquid-air interface. b, A tubular structure being gradually stretched, assembled, and grown as more ELPs and PAs assemble through the stretched membrane. c,d, Tubular networks being formed upon contact with a lateral surface, stretching, assembling, and growing. e. Merging of two tubular structures being formed next to each other.

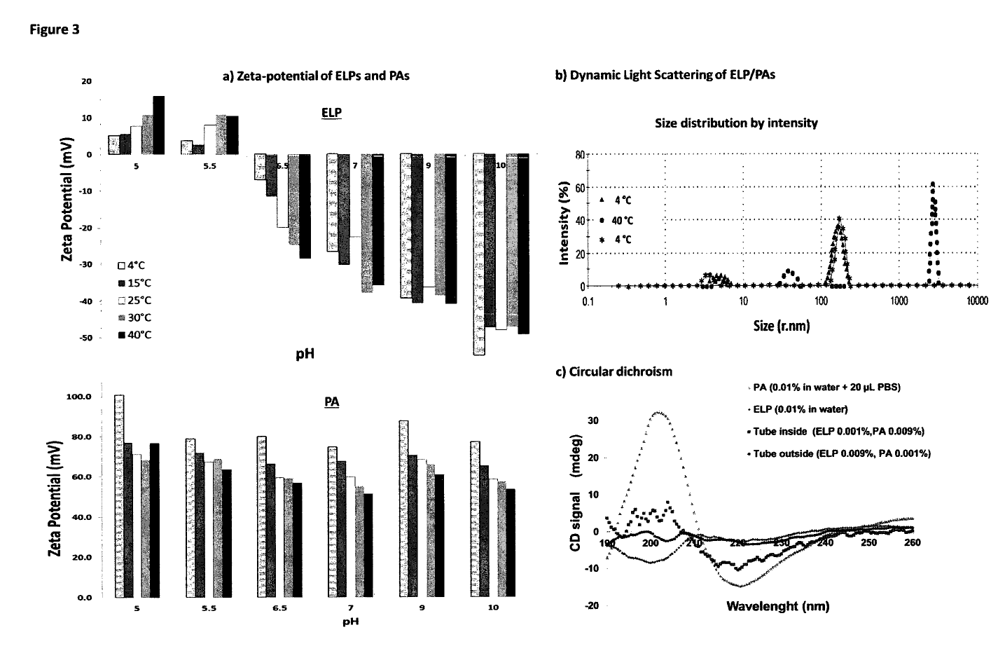

FIG. 3. Analysis of ELP and PA molecular interactions. a, Zeta potential measurements of ELPs and PAs at different pH and at different temperatures. b, Dynamic light scattering of ELP/PA mixtures above and below the ELPs' Tₜ. c. Circular dichroism of a solution of PAs and ELPs alone as well as mixtures aiming to recreate the environments present towards the inside (closer to the solution of PAs) and outside (closer to the solution of ELPs) of the tube.

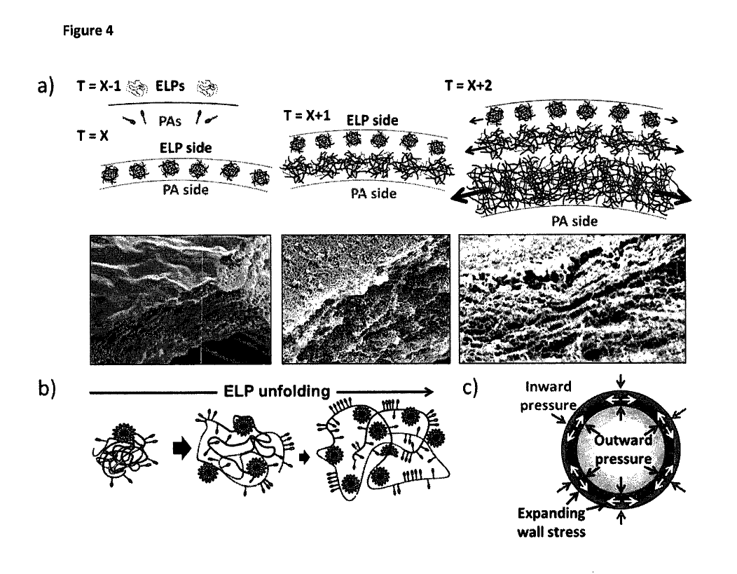

FIG. 4. Schematic of the proposed mechanism for the membrane formation. a, At the point of ELP-PA contact, as PAs begin to migrate towards the ELP side driven by electrostatic and hydrophobic forces, initial electrostatic interactions generate a thin membrane (T=X) formed by PA nanofibres and folded ELP chains. At T=X+1, the ELPs closer to the PA side begin to unfold due to hydrophobic bonding with PA molecules and towards the outside more ELPs begin to screen the charges of migrating PA molecules. This process results in the growing of the thickness of the membrane towards the outside. b, The ELP chains closer to the PA molecules begin to unfold as PA molecules bind through their hydrophobic tail, opening more spaces for PA nanofibre assembly and further PA molecule binding. This process generates a pre-stressed membrane with a higher concentration of ELP on the outside and a higher concentration of PAs inside. c, The resulting membrane experiences outward pressure from the migrating PAs, inward pressure from the ELP chains tending to move inside and the increased thickness of the membrane, and a wall-stress as a result of the unfolded chains.

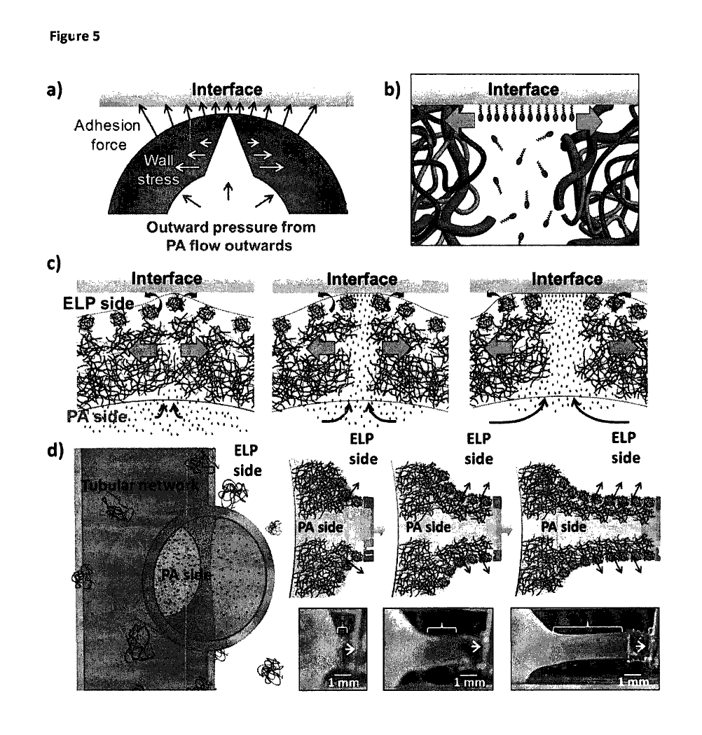

FIG. 5. Schematic of the proposed mechanism for opening of the membrane. a-c, membrane adhesion to the interface and consequent formation of a focal stress concentration point, which due to the pre-stress condition of the membrane crack propagates. As the membrane opens PAs flow towards and assemble on the interface further facilitating the opening of the membrane. d, Illustration of the process on the side wall. Following opening of the membrane, gradual stretching enables further ELP-PA interaction and co-assembly resulting in tubular networks.

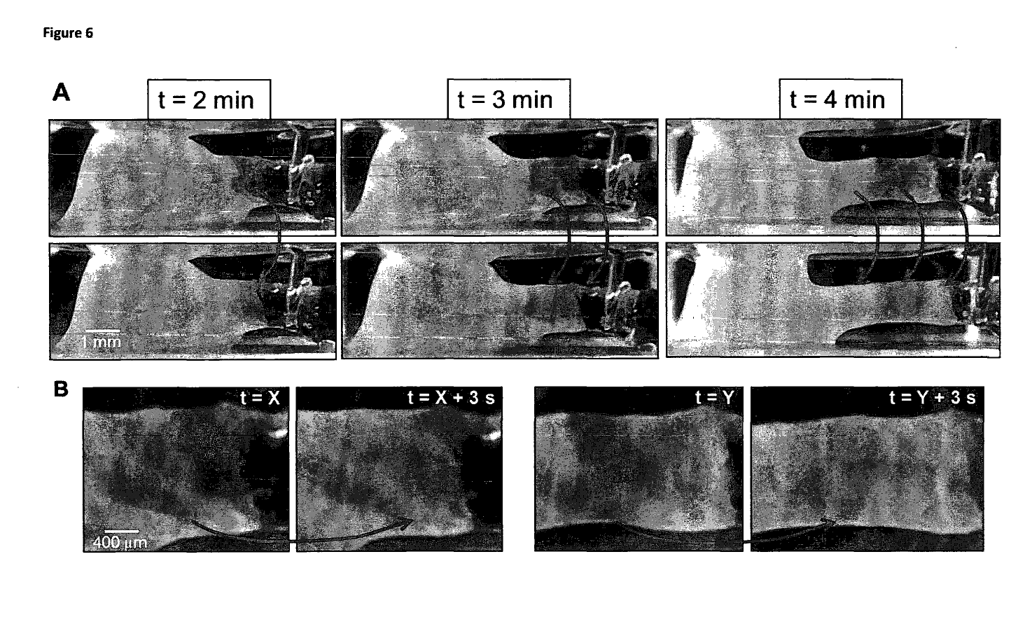

FIG. 6. Growing and self-healing properties of the material. a. Stretching of the tubes at higher rates could lead to distortions on the tube and rupture of layers within the walls. However, the walls exhibit self-healing capacity as PA molecules on the inside immediately interact with the ELP molecules on the outside. b. Close examination reveals the self-healing properties of the co-assembly system at different stages of a horizontal tube formation.

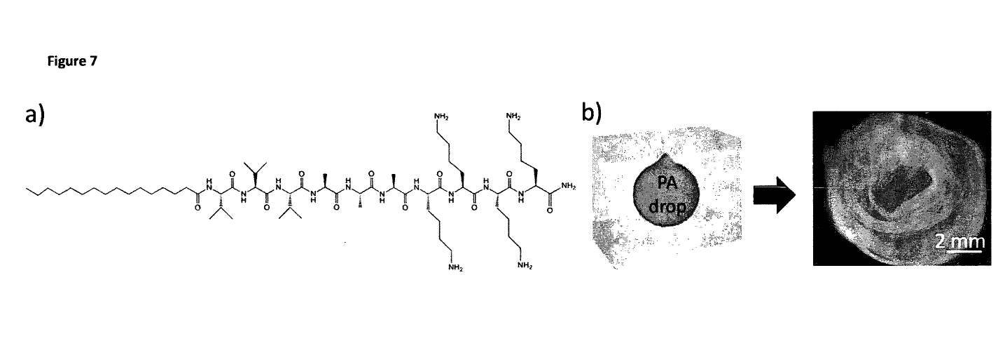

FIG. 7. a) Sequence of the peptide amphiphile K4 (PA-K4). b) Schematic representation of the methodology used for the membrane formation. Top view of ELP/PA-K4 tube formed by the standard tube formation protocol: 10 μl of 8.7 mM PA-K4 were added within 90 μl of ELP 0.1 mM. The tubes were allowed to form for 48 h.

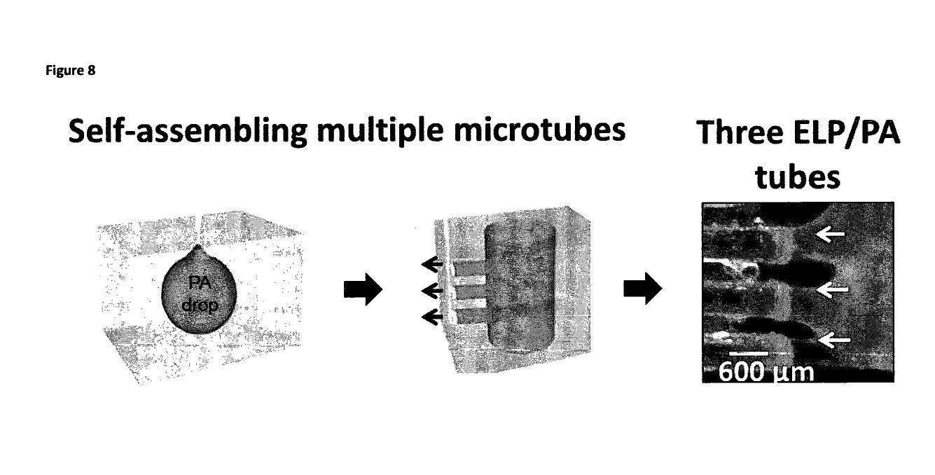

FIG. 8. Illustration of an ELP/PA self-assembled tubular structure with three sub-millimetre diameter tubes emerging from it.

Spatiotemporal control of molecular self-assembly for the fabrication of macroscopic peptide-based materials is major goal in many branches of materials science. Here, a multiple peptide system is shown that enables the possibility to direct molecular-self assembly at multiple scales on demand. The combination of elastin-like peptides (ELPs) with peptide amphiphiles (PAs) results in the formation of a dynamic membrane with the capacity to disassemble, be manipulated, and control the anisotropic co-assembly of ELPs and PAs to form complex macroscopic structures. The unique chemical-mechanical principles that dominate this system may also open the possibility to recreate those found in nature in tissue morphogenesis.

By simply placing a drop of an aqueous solution of a positively charged PA into a higher volume of negatively charged ELPs, a dynamic membrane self-assembles, which upon contact with any interface spontaneously forms hierarchical macrotubular structures (FIG. 1). The dynamic nature of the hybrid material permits the directed co-assembly of PAs and ELPs on demand into more complex macrostructures by simple manipulation. The resulting tubular macrostructures are robust and elastic, exhibited dynamic behaviour, directed post-assembly capabilities, the capacity to grow in size, and self-assemble into more complex geometries. This system may serve as a new material platform for the fabrication of complex macrostructures with hierarchical order and unprecedented tunability and morphogenic capabilities.

### Material Structure and Composition

The system incorporates a positively charged PA molecule and a negatively charged ELP with distinct hydrophobic segments in order to have a tunable system by both hydrophobic and electrostatic driving forces. At the interface between a solution of these molecules at specific conditions a solid membrane is formed upon their co-assembly (FIG. 1a-c). When a drop of the PAs is embedded inside a higher volume of a solution of ELPs, a solid closed membrane is instantaneously assembled, entrapping the PA solution inside and leaving the ELP solution outside. The closed membrane was observed to be sticky and undergo a remarkable transformation that permitted the spontaneous, yet controlled, opening of its surface upon contact with an interface within the first couple of minutes after co-assembly. In the simple case of initial membrane contact with either the air-liquid interface or the bottom surface, the closed membrane attached and opened to form a hollow half-sphere-like structure (FIG. 2a). When the closed membrane touched both interfaces a macrotube formed, which expanded between the two interfaces and exhibited a diameter that was similar to that of the initial drop of PA solution (FIG. 1f).

The membrane that made up the tube walls consisted of a network of nanofibres, which seemed to vary in both density and fibre morphology depending on their transverse position within the membrane (FIG. 1h). In addition, the outer part of the membrane was highly sticky while the inner part was not, which suggests an anisotropic composition with higher concentration of sticky ELP molecules outside and higher concentration of PA fibres on the inside. Elastin and ELPs have been found to bind to different surfaces probably due to their large hydrophobic segments. Time-of-flight secondary ion mass spectroscopy (ToF-SIMS), HPLC analysis, and enzyme specificity experiments confirmed this anisotropic structure. The tube membrane gradually turned from slightly cloudy immediately after mixing to denser white over the course of 48 h reaching a final thickness of about 20 μm as measured by scanning electron microscopy.

### Directed Self-Assembly on Demand

It was found that this unique hybrid membrane composition as well as its dynamic character enabled the possibility to direct further self-assembly on demand with high spatiotemporal control. By increasing the volume of the ELP solution, the tubes were observed to stretch slightly as their ends were attached to both the bottom surface and the liquid-air interface. The stretched region appeared more translucent but immediately began to turn white, suggesting that further ELP-PA co-assembly was taking place. It is likely that as the stretched network of fibres that make up the walls rearrange, newly opened spaces between the fibres facilitate further flow and interaction between the internal PA solution (which acts as a reservoir of PAs) and the external one (which serves as a reservoir of ELPs). Interestingly, reiteration of this process allowed us to sequentially stretch, assemble, and grow the tubular macrostructures by at least 300% over 2 h (FIG. 2b).

The controlled manipulation of this extraordinary dynamic behaviour enables the possibility to trigger self-assembly on demand, hierarchically, and with high spatiotemporal regulation. For example, by touching the lateral walls of newly formed tubes, it was possible to create openings on their sides by the same mechanism. By repeating the gradual stretch/assembly/growth technique, new tubular structures emerged in the direction of the moving interface, enabling the possibility to reproducibly create tubular networks of various sizes and shapes (FIG. 2c,d). Moreover, by controlling the area of the touching interface, it is possible to modulate the diameter and quantity of the tubes (FIG. 2d). For example, using a microfabricated structure comprising post topographical patterns, multiple microscopic tubes can be grown. In this way, this system enables both controlled co-assembly at the molecular level and guidance of subsequent growth at the micro and macroscopic scales to generate hybrid, complex, self-assembled tubular networks. In addition, when a second drop of PA was placed next to a newly formed tube within the ELP solution, the tubes adhered and within 30 min gradually merged forming one larger tube (FIG. 2e).

After 48 h the macrostructures could easily be handled with tweezers and exhibited considerable strength and elasticity both in water and air (FIG. 1g). Moreover, qualitatively the tubes strengthen markedly within the first 48 h. This increase in strength along with the change in appearance over time confirm that membrane assembly and tube formation are dynamic processes.

### Mechanisms of Formation

It is well established that electrostatic and hydrophobic interactions play a critical role in both PA self-assembly and ELP folding above its Tₜ. Indeed, independently of the mixing conditions, visible gel-like aggregates always resulted upon mixing the PAs with the ELPs. However, membrane opening and formation of the tubular structures were highly dependent on a number of critical factors including experimental setup, molecule concentration, pH, time, temperature, and peptide sequence and design. For a better interpretation of the mechanisms behind the observed ELP-PA tubular morphogenesis, we divide the processes into three stages including a) initial membrane formation, b) membrane growth, and c) membrane opening and tubular growth.

a. Initial Membrane Formation

Thin solid membranes instantaneously form when the two liquids enter into direct contact above the ELP's Tₜ (15° C.). We believe this initial membrane is primarily induced by the expected electrostatic charge screening of the positive PAs by the negative segments of the ELPs above pH 6. Below pH 5.5 the ELP was seen to be slightly positive owing to the protonation of the two arginine residues and neutralization of a large proportion of the carboxylic acids, which disrupted the interaction between the two molecules. In contrast, robust membranes formed when both ELPs and PAs had stronger opposite zeta potentials (FIG. 3a), as has been previously demonstrated using PAs and surfactants in combination with oppositely charged polyelectrolytes. However, in this case, tubes only formed when the pH was between 6 and 7.5 and the zeta potential of both molecules was marginally opposite. In other words, the membrane opening did not occur when the two molecules had strongest opposite charge, suggesting that the dynamic and versatile nature of our ELP-PA system depends on an initial membrane that is formed by intermediate electrostatic forces. To test this hypothesis, the procedure was repeated using similar PAs but with 2 lysine residues, resulting in closed membranes that did not open and 4 lysine residues resulting in unstable tubes.

Under these conditions, this initial membrane begins to serve as a semipermeable barrier across which the small PA molecules begin to diffuse towards the ELP side interacting both electrostatically and hydrophobically with ELPs. This PA transport is credited to a diffusion-like drift arising from a PA chemical potential (concentration) gradient as well as an osmotic pressure imbalance resulting from the initial concentration difference between the PA (8.696 mM) and the ELP (0.089 mM) solutions. This initial stage is comparable to previously described sac structures and similarly may be modeled by the Smoluchowski transport equation formulated as a Donnan-like term gauged in terms of the zeta potential difference between the two solutions.

$$Δπ = {π}_{PA} - {π}_{ELP} = {ζ}^{2} Σ \frac{\text{?}}{} > 0$$

$$\text{?} \text{indicates text missing or illegible when filed}$$

b. Membrane Growth

After this initial interaction, hydrophobic forces begin to play a crucial role. Below the ELP's Tt, weak hydrogel-like structures were formed, which are likely a result of electrostatic screening of the PAs that trigger them to self-assemble into the classical well-defined nanofibres. Above the Tₜ the ELPs undergo dehydration of their hydrophobic segments and subsequently fold into their characteristic β-spiral conformation, exposing large non-charged hydrophobic segments. As PAs diffuse, they begin to assemble into more nanofibres and orient/attach their alkyl tails directly onto these large hydrophobic sections of the ELP chains (FIG. 4a,b). In order to test for this hypothesis. DLS measurements were conducted on a combined solution of the two molecules at pH 7. Below the Tₜ, the solution exhibited aggregates of about 100 nm in size, while above the Tₜ aggregates increased up to 2.6 μm (FIG. 3b). In addition, cooling of formed tubes below the Tₜ resulted in significant weakening of the structures due to hydration of the folded ELP chains. Furthermore, using similar PAs but with reduced alkyl tails of 6 Carbons instead of 16, only resulted on weak hydrogel-like aggregates. All together, these results demonstrate the key role of the hydrophobic interactions in the ELP-PA system.

As PAs continue to diffuse and bind in higher concentrations on the ELPs, they begin to open the ELPs' highly folded conformation. This kind of surfactant-induced unfolding is well documented for globular proteins. As the ELP chains unfold, a different physico-chemical mechanism begins to assume the kinetic control of the process and two simultaneous events take place. First, the ELP-PA membrane becomes more permeable and exposes more interaction active sites, which in turn drive more PA recruitment, PA nanofibre assembly, and PA-ELP hydrophobic interactions that further promote additional ELP unfolding. Second, unfolding of ELP chains and formation of more PA nanofibres generate a PA-driven volume expansion and consequently a crucial pre-stress state on the innermost layers of the membrane. The situation is thus viewed as a swelling gel, with the ELPs (expanding with more and more PAs) playing the role of the elastic component of the gel and the fresh PAs (assembling or binding within the membrane) playing the role of the solvent. Within this scenario, the initial ELP/PA membrane would transform progressively into a multilayer structure separating the two liquids with the inner layers being richer in unfolded ELP chains and PAs (both in nanofibre form and hydrophobically bound to the ELPs).

The proposed membrane structure is illustrated in FIG. 4a and may be described as a nanofibrous hybrid with high concentration of PAs towards the inner part of the membrane and a high concentration of ELPs towards the outside. It is likely that fibres on the inside of the membrane are mostly PA nanofibres resulting from the higher proportion of PA molecules. However, on the outside surface it is likely that the observed nanofibres are structurally different, as they would contain a much higher proportion of ELPs. It is known that globular proteins can undergo surfactant induced fibrillation at specific concentrations and ELPs can generate elastin-like aggregates above the Tt. This anisotropic structure is also evidenced by the observed highly sticky outside surface and the differences in fibre morphology of the inner and outer surfaces (FIG. 1h). Furthermore, disassembly of the tube structure primarily on the outside surface upon cooling below the ELPs' Tt, indicates the presence of an increased concentration of ELPs on this side of the membrane. In order to investigate these differences in more detail, the environments of the inside and outside of the tubes were roughly simulated by combining a small volume of one molecule with a large volume of the other, both at low concentration. Circular dichroism (CD) analysis confirmed that the conditions recreating the environment inside of the membrane result in higher β-sheet contribution, while those recreating the outside are mainly characterized by a random coil conformation, supporting the hypothesis that the majority of fibres on the outside are not classical PA nanofibres. This anisotropic structure was further confirmed by using fluorescently-labelled PA molecules and observing higher fluorescence towards the inside of the membrane where higher concentration of PAs would be expected.

c. Membrane Opening and Tubular Growth Opening of the membrane occurs at any point within the first couple of minutes after mixing upon touching an interface. Within this short timeframe, the forming membrane is highly unstable, similarly to what occurs when a gel undergoes swelling under an imposed volume restriction. Since layers are stiffer the more exterior they reside within the membrane, the system is subjected to a constrained stress distribution that results from the outward pressure from migrating PA molecules, the inward pressure from the stiffer outer layers of the membrane, and the critical expanding wall stresses (swelling) that result from the unfolding ELP chains of the inner layers of the membrane (FIG. 4b, c, 5a). The natural time scale of this stage can be obtained from the theory of swelling microgel shells. Our model describes the internal motion undertaken by the gel network to relax its mechanical stress. The equation for the displacement of an interior point in the network is

$$ρ \frac{{\partial }^{2} u}{\partial {t}^{2}} = \overline{v} , σ - f \frac{\partial u}{\partial t}$$

where ρ is the density of the medium, σ stands for the stress tensor, and ƒ denotes a friction coefficient between ELP/PA and pure PA. In addition, considering the boundary conditions for a swelling spherical gel, the displacement vector is spherically symmetric and described by

u(r,t)=u(r,t)r/r

while the radial component of the stress is

σᵣᵣ=(K+4μ/3)(du/dr)+2(K-2μ/3)(u/r)

where K and μ correspond to the bulk and shear modulus, respectively, of the gel network (composite ELP/PA material in this case). Neglecting the inertia term, this theoretical scheme results in an eigenfunction problem for the diffusion operator

$$\frac{\partial u}{\partial t} = {D}_{0} \frac{\partial }{\partial r} \{ \frac{1}{{r}^{2}} \vert \frac{\partial }{\partial r} ( {r}^{2} u ) \vert \}$$

The sought time scales are found in terms of an effective diffusion of the gel network D₀ and the eigenvalues λₙ of the corresponding operator. The coefficient D₀ can be expressed as D₀=(K+4μ/3)/ƒ. The set of eigenvalues is more difficult to obtain since the eigenvalue problem turns out to be non-Hermitian. In any case, the relevant length corresponding to the dominant eigenvalue scales with the droplet radius. Thus an independent estimation of the moduli of the ELP material, whether feasible, could easily predict the swelling time constant.

With this in mind, during the initial moments after mixing, the highly unstable membrane would likely develop a buckling instability over the whole droplet surface. If the membrane touches an interface during this early time point, the highly sticky ELP molecules located on the outer surface of the closed membrane will stick and develop a cusp that creates a focal point of stress concentration on the pre-stressed membrane, resulting in a tendency to rupture the packed internal conformation and crack propagate (FIG. 5a-c). Simultaneously, the fresh PA molecules in solution will be freely exposed to the interface and tend to spread on it given its surfactant nature. Meanwhile, the interface ring that surrounds the newly formed neck would accommodate accumulated ELP/PA material as it is pushed radially outwards from the opening point. Close examination of the ends of formed tubes revealed a “sleeve” (FIG. 1e,g,2a), which is likely this accumulated and dragged material.

Although PA spreading on an interface can be considered an entropically penalized process it would be favored from the naturally existing lateral gradient of surface pressure. More precisely, PA molecules residing at this free surface would tend to expand their occupying area through the usual surfactant based principle of reduction in the surface tension: γ_{air/PA}−γ_{air/ELP}<0. This expanding neck process is however very slow in our situation since the PA occupied area is not expanding against a bare surface but rather against the ELP-based sticky outer layers of the membrane. This ELP-based sealed environment is essential to prevent mixing of the two solutions and enable the controlled manipulation and remarkable morphogenesis of the system.

As explained earlier, ELP-PA tube structures are able to grow longitudinally because of the rearrangement of fibres that in the additional ELP-PA interactions (FIG. 5d). If the rate of stretching is higher than that of the ELP-PA interactions, the tube will tend to decrease in diameter (FIG. 2b). At even higher stretching rates, cracks on portions of the membrane may appear (FIG. 6). However, immediate flow of ELP from the outside and PA from the inside would spontaneously heal and regenerate the membrane within a few minutes (FIG. 6b). Interestingly, at these high strain-rates, the walls of the tubes do not crack open completely but rather seem to brake at different layers within the membrane (FIG. 6b). This observation supports the hypothesis that the cross-section of the membrane is made from many molecular layers experiencing different wall stresses as a result of their specific ELP-PA composition as described earlier (FIG. 4a).

It is believed that the interactions between mechanical and chemical elements of the membrane are central to the remarkable capacity of the system to undergo morphogenesis. This behaviour has enabled the possibility to direct self-assembly on demand and fabricate complex macroscopic structures by ELP-PA co-assembly. Interestingly, this dynamic process may be compared to that taken place in biology, where an interplay of mechanical and chemical aspects are known to play a major role during development. For example, in elastic tissues anisotropic growth is largely dependent on mechanical effects due to spontaneous instabilities. In addition, morphogenesis during epithelial development is modulated by focal defects that lead to local tensional forces in the basement membrane.

### CONCLUSION

The discovery of a peptide/protein-like system that enables the possibility to direct molecular-self assembly at multiple scales on demand has been demonstrated. The key feature is the generation of an ELP-PA hybrid and anisotropic membrane that is highly unstable during its dynamic formation. During the early part of its formation, its careful manipulation enables the controlled guidance of further ELP-PA co-assembly with remarkable temporal and spatial control into macrotubular networks. The resulting macrostructures are robust and elastic, exhibit dynamic behaviour, directed post-assembly capabilities, self-healing properties, and the capacity to grow and self-assemble into more complex tubular geometries without the use of predefined moulds or templates. This work introduces a novel material platform to create complex 3D macroscopic structures for a variety of potential applications that may range from the generation of vascular grafts or peripheral nerve conduits to the fabrication of biosensors and electronic devices. The principles that dominate this system may also open the possibility to recreate those found in nature in tissue morphogenesis.

1. ELP/PA Membrane Formation and Characterisation

The ELP used in the example above is a pentablock structure—ELP pentablock in the table below. Other ELPs were also used in this work and are described below, along with some other molecules.

| | Sequences | Name |
|---|---|---|
| Protein | | |
| Elastin-Like Polymer | MESLLP-[(VPGVG VPGVG VPGEG | ELP |
| Pentablock | VPGVGVPGVG)₁₀-(VGIPG)₆₀]₂-[(VPGIG)₁₀- | |
| | AVTGRGDSPASS(VPGIG)₁₀]₂V (SEQ ID NO: 8) | |
| Elastin-Like Polymer | MESLLP-[(VPGVG VPGVG VPGKG | ELP4B |
| Tetrablock | VPGVGVPGVG)₁₀-(VGIPG)₆₀]₂-V (SEQ ID NO: 9) | |
| Elastin-Like | (VPGIG VPGIG VPGKG VPGIG VPGIG)₂₄ | ELP-IK |
| Polymer IK | (SEQ ID NO: 10) | |
| Elastin-Like | [[(VPGIG)₂(VPGKG)(VPGIG)₂]₂AVTGRGDSPASS | ELP-RGDS |
| Polymer RGDS | [(VPGIG)₂(VPGKG)(VPGIG)₂]₂]₆ (SEQ ID NO: 7) | |
| Glycan | | |
| Heparan Sulphate | Succession of (GlcA-GlcNAc)ₙ | HS |
| Chondroitin Sulphate | Succession of (GlcA-GalNAc)ₙ | CS |
| Peptide name | | |
| Peptide Amphiphile 1 | C16-VVVAAAKK (SEQ ID NO: 11) | PA-K2 |
| Peptide Amphiphile 2 | C16-VVVAAAKKK (SEQ ID NO: 12) | PA-K3 |
| Peptide Amphiphile 3 | C16-VVVAAAKKKK (SEQ ID NO: 13) | PA-K4 |
| Peptide Amphiphile 4 | C16-VVVAAAEEE (SEQ ID NO: 14) | PA-E3 |
| Peptide Amphiphile 5 | C16-KKK | C16-K3 |

### Negative Molecules and PA-K3 Interaction

Instead of the ELP molecules, other negative molecules including Bovine Serum Albumin (BSA) and sodium dodecyl sulfate (SDS) were mixed with PA-K3 under the same procedure used for the ELP/PA-K3 disclosed above. BSA at 0.1 mM concentration was mixed with PA-K3 at 8.7 mM at a ratio 9:1. An interaction between BSA and PA-K3 was observed leading to the formation of a membrane at the point of contact between the two solutions. However, no tubular architecture was obtained. On the other hand, SDS at 0.1 mM was mixed with PA K3 at 8.3 mM at a ratio 9:1. In this case, no visible interaction was seen between these two molecules.

### Surfactant Effect of PA-K3

PAs are surfactant-like molecules. In order to better understand the interaction between ELPs and PAs, the commercially available positive surfactant DodecylTrimethylAmmonium Bromide (DTAB) at 0, 9, 18 and 20 mM was mixed at a ration 9:1 with ELP negatively charged at 0.1 mM. No visible interaction was detected. However, at higher DTAB concentrations, a visible interaction between both molecules was observed but no membrane was formed. In order to compare the effect of PA and DTAB on ELP molecules, electrophoretic mobility and dynamic light scattering measurements were performed. Results showed that PA and DTAB have a similar effect on the mobility and the size of ELP molecules. These results support the hypothesis that electrostatic forces are not the only forces involved during the ELP/PA membrane formation.

### Tubular Structure Formed Using Another Sequence of Peptides Amphiphile (PA)

ELP/PA membranes similar to the membranes previously described in the application can be formed using an alternative PA sequence: C16-V₃A₃K₄ (PA-K4—SEQ ID NO: 13; FIG. 7a). The structures were formed using the same procedure than that of ELP/PA-K3 membranes i.e. at 1 mM was mixed with PA-K4 at 8.7 mM on a hydrophobic surface at a ratio 9:1 (FIG. 7b). The membrane formed opened in tubular structures with similar properties to that of ELP/PA-K3 membranes.

Other PA and ELP Sequences The mixture of positively charged PA (PA-K3) with either ELP-IK or ELP-RGDS did not result in visible macroscopic structures. Similarly, negatively charged (PA-E3) and negatively charged ELP did not result in visible interactions. However, when a negative PA molecule (PA-E3) was mixed with a positively charged ELP (ELP-IK or ELP-RGDS), stable gel-like interactions were achieved although a no tubular structures were obtained. Similarly, when another positive PA-K2 (C16-V₃A₃K₂—SEQ ID NO: 11) was mixed with the negative ELP, a membrane was formed but no tubular structure was obtained. Instead, a closed membrane structure was formed. Overall, these results underline the importance of the sequences used to form these tubular structures. ELP/PA interaction may be essential for the formation of the tubular membrane structure and may be driven by an association of electrostatic forces (oppositely charges of the PA and ELP molecules), hydrophobic forces, adhesion forces between the ELP molecules and any surface, and the generation of an internal wall-stress.

### Characterisation of the ELP/PA-K3 Membrane

Infrared spectrum was obtained on ATR-FTIR spectrometer. Differences were observed between the inner part and the outer part of the membrane. A higher number of amide bonds was observed in the inner part of the membrane than in the outer part of the membrane.

2. Membrane Stabilisation and Functionalization

Stabilisation of the Membrane

Different ways to cross-link the hybrid ELP/PA-K3 have been explored to improve on its stability, which may be required depending on the application. Tube structures have successfully been cross-linked using either GTA. EDC/NHS. SCM-PEG-SCM. 4S-StarPEG or Genipin. Once the membrane has been formed for 48 h, the membrane was washed with water to remove the excess of PA-K3 and ELP molecules. ELP/PA-K3 constructs were then cross-linked with 1 mM poly(ethylene glycol) ether tetrasuccinimidyl glutarate (4S-StarPEG), 1 mM (Succinimidyl Carboxymethyl Ester)₂ (SCM-PEG-SCM) (JenKem Technology. USA), 3 mM Genipin (Cambridge Bioscience. UK), 0.625% glutaraldehyde (Sigma Aldrich, UK) and 3 mM EDC/NHS (Sigma Aldrich, UK) diluted in water for 1 h at 37° C. The non-cross-linked tubes became weaker when immersed in PBS 1× after 24 h while the stabilised structures in PBS 1× were stabled in PBS for at least 14 days.

### Addition of Proteins and Glycans

Silk fibroin protein can be incorporated within the membrane to improve functionalization and complexity. Silk fibroin protein at a concentration of 0.1 mM was mixed with an equal quantity of ELP (ratio 1:1). No interaction between ELP and silk fibroin protein was observed. Similarly to ELP protein alone, a drop of 90 μl of silk/ELP mixture was deposited on a PDMS surface. 10 μl of positive PA-K3 at 8.3 mM was added in the drop and incubated at 21° C. for 48 h. A tubular structure was obtained.

Similarly, heparan sulphate (HS) and chondroitin sulphate (CS), glycans of the extracellular matrix, can be incorporated within the membrane during the assembly process and will also create tubular structures. HS at a final concentration of 0.5 mg/mL was mixed with a solution of ELP at 0.1 mM. No interaction between HS and ELP was observed. Similarly to ELP protein alone, a drop of 90 μl of HS/ELP mixture was deposited on a PDMS surface. 10 μl of positive PA-K3 at 8.3 mM was added in the drop and incubated at 21° C. for 48 h. A tubular structure was obtained. Tubular structures were obtained with an increased concentration of HS. The incorporation of glycans within the membrane also increased the stability of the membranes in salt even without chemical cross-linking. Indeed, membranes were observed stable in PBS 1× for at least 14 days. It also allows a functionalization of the membrane. Similar observations were made after addition of CS.

3. Cell Adhesion

Cells, such as mouse adipose-dericed stem cells (ADSCs) adhered and grew on the ELP/PA constructs cross-linked with 1 mM poly(ethylene glycol) ether tetrasuccinimidyl glutarate (4S-StarPEG) beforehand 50.000 cells/cm² were seeded on the membrane in DMEM (Glutamax™, Life Technologies, UK) supplemented with 10% fetal bovine serum and 1% of penicillin/streptomycin (Sigma Aldrich, UK). Seeded constructs were incubated at 37° C. under 5% CO₂ for 24 h. After 24 h, a Live-Dead Assay® was conducted according to the manufacturer's instructions (Life Technologies, UK). Alive cells were stained with calcein (green) while dead cells were stained with Ethidium Bromide (Red). Most of the cells observed adhered on the constructs were viable while a little number of dead cells were seen.

4. Tube Embedded within a Hydrogel

ELP/PA tubes can be self-assembled and embedded within a hydrogel such as a crosslinked type I atelocollagen hydrogel. After formation of the tube as previously described for 48 h. the tubes were washed to remove any excess of PA and ELP. The pH of a solution of type I atelocollagen, dissolved in 0.05M acetic acid at a concentration of 3 mg/mL, was adjusted to 7.4 using 1M NaOH and 5× Phosphate Buffer Saline (PBS) for obtaining a final concentration of 1 mg/mL. 4S-StarPEG was then added to final concentrations of 1 mM. This solution was immediately poured around the ELP/PA constructs. The constructs were incubated for 1 h at 37° C. to induce gelation. The constructs were stable after incubation in PBS 1× for at least one week.

5. Multiple Microtubes

Following a similar procedure as that described above, further assembled tubular structures with more complex shapes have been produced, including multiple sub-millimetre diameter tubes (FIG. 8).

### REFERENCES

1. Castellano, V., Hamley, I. W., Adamcik, J., Mezzenga, R., Gummel, J. Modulating self-assembly of a nanotape-forming peptide amphiphile with a oppositely charged surfactant. Soft Matter, 2012, 8, 217.
2. Andersen, K. K., et al. The role of decorated SDS micelles in sub-CMC protein denaturation and association. J. Mol. Biol. 391, 207-226 (2009).
3. Giehm. L., Oliveira, C. L. P., Christiansen. G., Pedersen, J. S., Otzen. D. E. SDS-induced fibrillation of α-synuclein: an alternative fibrillation pathway. J. Mol. Biol. 401, 115-133 (2010).

1. A composition comprising a self-assembling membrane, wherein the self-assembling membrane comprises elastin-like polymers (ELPs) and peptide amphiphiles (PAs).

2. The composition of claim 1, wherein the ELPs are negatively charged and the PAs are positively charge.

3. The composition of claim 1, wherein the ELPs comprise a polymer of the pentamer VPGVG and/or a pentamer in which one or more amino acids have been substituted by another amino acid.

4. The composition of claim 3, wherein the pentamers are selected from VPGVG, VPGDG, VPGEG, VGIPG and VPGIG.

5. The composition of claim 3, wherein the ELPs comprise a polymer comprising, as monomers, the pentamers VPGVG, VPGEG, VGIPG and VPGIG.

6. The composition of claim 5, wherein the polymer contains the pentamers in the following ratios relative to each other:
a) VPGVG: 70-90;
b) VPGEG: 10-30;
c) VGIPG: 110-130; and
d) VPGIG: 30-50.

7. The composition of claim 1, wherein the ELPs comprise a bioactive domain.

8. The composition of claim 1, wherein the molecular weight of the ELPs range between about 10 kDa and about 250 kDa.

9. The composition of claim 1, wherein the ELPs have an average molecule weight of between about 10 kDa and about 50 kDa.

10. The composition of claim 1, wherein the PAs comprise a peptide sequence with an alkyl tail having 10-20 carbon atoms.

11. The composition of claim 1, wherein the PAs comprise a peptide sequence having between 7 and 11 amino acids.

12. The composition of claim 1, wherein the membrane is crosslinked.

13. The composition of claim 1, wherein the membrane is in the form of a tube.

14. The composition of claim 13, wherein the tube has two or more openings.

15. The composition of claim 1, wherein the membrane further comprises silk fibroin protein or a glycan.

16. The composition of claim 15, wherein the glycan is heparan sulphate or chondroitin sulphate.

17. A method of forming a membrane, the method comprising contacting ELPs and PAs, wherein interaction between the ELPs and PAs causes formation of the membrane.

18. The method of claim 17, wherein the ELPs and PAs are in aqueous solution such that separate solutions containing each of the components are brought together to contact the ELPs and PAs.

19. The method of claim 17, wherein a solution of PAs is added to a larger volume of an ELP solution.

20. The method of claim 18, wherein the PA solution has a concentration of between about 0.5 wt % and about 3 wt %.

21. The method of claim 18, wherein the ELP solution has a concentration of between about 0.5 wt % and about 1.5 wt %.

22. The method of claim 18, wherein the membrane is formed at a pH of between about 5 and about 8.

23. The method of claim 18, wherein the membrane is formed at a temperature which is greater than the transition temperature (Tt) of the ELPs.

24. The method of claim 18, wherein the PAs and ELPs are allowed to interact for 12 hours or more.

25. The method of claim 18, wherein the membrane is manipulated during formation so as to control the shape into which it forms.

26. The method of claim 18, wherein the method further comprises the step of crosslinking the membrane.

27. A kit for forming a self-assembled membrane, the kit comprising ELPs and PAs.


SEQUENCE LISTING (14 sequences)

SEQ ID NO: 1 - protein, 5 aa, Artificial sequence
  Repeating pentapeptide sequence from elastin- like peptides (ELPs)
VPGXG

SEQ ID NO: 2 - protein, 5 aa, Artificial sequence
  Repeating pentapeptide sequence from elastin- like peptides (ELPs)
VPGVG

SEQ ID NO: 3 - protein, 5 aa, Artificial sequence
  Repeating pentapeptide sequence from elastin- like peptides (ELPs)
VPGDG

SEQ ID NO: 4 - protein, 5 aa, Artificial sequence
  Repeating pentapeptide sequence from elastin- like peptides (ELPs)
VPGEG

SEQ ID NO: 5 - protein, 5 aa, Artificial sequence
  Repeating pentapeptide sequence from elastin- like peptides (ELPs)
VGIPG

SEQ ID NO: 6 - protein, 5 aa, Artificial sequence
  Repeating pentapeptide sequence from elastin- like peptides (ELPs)
VPGIG

SEQ ID NO: 7 - protein, 672 aa, Artificial sequence
  Elastin-like peptide (ELP) sequence
VPGIGVPGIGVPGKGVPGIGVPGIGVPGIGVPGIGVPGKGVPGIGVPGIGAVTGRGDSPASSVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGAVTGRGDSPASSVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGAVTGRGDSPASSVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGAVTGRGDSPASSVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGAVTGRGDSPASSVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGAVTGRGDSPASSVPGIGVPGIGVPGKGVPGIGVPGIGVPGIGVPGIGVPGKGVPGIGVPGIG

SEQ ID NO: 8 - protein, 1331 aa, Artificial sequence
  Elastin-like peptide (ELP) sequence
MESLLPVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVPGIGVPGIGVPGIGVPGIGVPGIGVPGIGVPGIGVPGIGVPGIGVPGIGAVTGRGDSPASSVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGVPGIGAVTGRGDSPASSVPGIGVPGIGVPGIGVPGIGVPGIGVPGIGVPGIGVPGIGVPGIGVPGIGV

SEQ ID NO: 9 - protein, 1107 aa, Artificial sequence
  Elastin-like peptide (ELP) sequence
MESLLPVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGV

SEQ ID NO: 10 - protein, 1706 aa, Artificial sequence
  Elastin-like peptide (ELP) sequence
MESLLPVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVPGVGVPGVGVPGEGVPGVGVPGVGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVGIPGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIG

SEQ ID NO: 11 - protein, 8 aa, Artificial sequence
  Sequence of proteinous part of peptide amphiphile (PA)
VVVAAAKK

SEQ ID NO: 12 - protein, 9 aa, Artificial sequence
  Sequence of proteinous part of peptide amphiphile (PA)
VVVAAAKKK

SEQ ID NO: 13 - protein, 10 aa, Artificial sequence
  Sequence of proteinous part of peptide amphiphile (PA)
VVVAAAKKKK

SEQ ID NO: 14 - protein, 9 aa, Artificial sequence
  Sequence of proteinous part of peptide amphiphile (PA)
VVVAAAEEE